(19) United States
(12) Patent Application Publication
(10) Pub. No.: US 2016/0270826 A1
(43) Pub. Date: Sep. 22, 2016

(54) BONE ANCHORING ASSEMBLIES AND METHODS OF USE

(71) Applicant: Trinity Orthopedics, LLC, San Diego, CA (US)

(72) Inventors: JAMES F. MARINO, La Jolla, CA (US); Jamil Elbanna, San Diego, CA (US)

(21) Appl. No.: 15/074,900

(22) Filed: Mar. 18, 2016

Related U.S. Application Data
(60) Provisional application No. 62/135,973, filed on Mar. 20, 2015.

(51) Int. Cl. A61B 17/70

(52) U.S. Cl. CPC A61B 17/7037; A61B 2017/8655

(57) ABSTRACT

A fixation system is described that can be secured to a bony structure, such as a pedicle of a vertebrae. For example, the fixation system can include a pedicle screw and a receiver element. The receiver element can have a pair of upper opposing projections with a first channel formed therebetween and shaped to receive a part of a connecting rod. The receiver element can further include a pair of lower opposing projections with a second channel formed therebetween and shaped to mate with the pedicle screw. The fixation system can further include an expanding element that forms a tubular shape when a compressive force is applied along a plurality of flexible arms of the expanding element. The plurality of flexible arms can be self-expanding and bow radially outward when the compressive force is released.

### CROSS-REFERENCE TO RELATED APPLICATION

The current application claims priority under 35 U.S.C. §119(e) to U.S. Provisional patent application Ser. No. 62/135,973, filed on Mar. 20, 2015, and entitled “Bone Anchoring Assemblies and Methods of Use,” which is incorporated by reference herein in its entirety.

### FIELD

The present disclosure relates generally to orthopedic boney fixation systems and methods, and more particularly, to pedicle fixation systems and methods.

### BACKGROUND

Pedicle screws are a mainstay of spine fixation due to the relatively superior mechanical association they provide in associating with vertebrae relative to other forms of fixation (e.g. interspinous process fixation, facet fixation or vertebral body plates). The quality of fixation is dependent upon a number of variables, including the relative diameter of the screw with respect to the pedicle, the length of screw purchase, and the design of the screws thread form. However, none of the aforementioned variables determines the pedicle screw's strength of purchase more than the density of the bone within which the pedicle screw resides.

Osteopenia and osteoporosis are conditions of bone in which the density of bone matrix (protein and mineral content) are reduced relative to youthful and healthy norms (the latter being a more severe loss of bone mass than the former). These conditions evolve as a normal consequence of aging in a large percentage of the adult population, with most women and men experiencing a progressive decline in their bone density from their mid-twenties on. Due to greater initial bone mass, men experience clinical consequences of osteopenia (low energy fractures) about ten years later than their female counterpart. Postmenopausal women have a very high incidence of osteopenia (defined as more than one standard deviations of reduced bone density relative to a youthful normative value) and osteoporosis, the more severe form of the condition (defined as two and one half standard deviations or more of reduced bone density relative to a youthful normative value). As an indication of its prevalence in postmenopausal women, one in three women will sustain a reduced bone density related fracture, by age sixty. Men over the age of fifty have an incidence of osteopenia of approximately 30 to 45%, with half of octogenarian men having osteoporosis.

Spinal fixation procedures are frequently indicated in elderly patients who suffer from clinical syndromes that include degenerative spondylolisthesis, degenerative scoliosis, and spinal stenosis. In addition, the propensity for pedicle screws to dislodge from their boney attachment increases dramatically in those patients requiring “long construct fixation” or pedicle screw fixation in more than a few spine segments. Proximal junction kyphosis (PJK) is a well-recognized complication of long construct surgery for which limited ameliorating interventions are available.

Pedicle screw fixation can be increased with the instillation of methyl methacrylate, or bone cement into the vertebra prior to pedicle screw insertion. There are significant potential adverse effects from the use of bone cement in this application, due to thermal necrosis of the bone resulting from the exothermic reaction of curing, the potential of cement embolization, the potential for bone cement leaking into adjacent areas (e.g. the foramina, spinal canal, or disc space) and diminished vascular nutrition for the vertebra and adjacent disc space.

### SUMMARY

These and other aspects will now be described in detail with reference to the following drawings. Generally speaking the figures are not to scale in absolute terms or comparatively but are intended to be illustrative. Also, relative placement of features and elements may be modified for the purpose of illustrative clarity.

Aspects of the current subject matter can include a fixation system that can be secured to a boney structure. For example, an implementation of the fixation system can include a bone screw and a self-expanding element configured to be associated with the bone screw. The self-expanding element can have a narrow constrained configuration during delivery and an expanded deployed configuration after delivery that increases resistance of the bone screw to displacement. In some variations one or more of the following features can optionally be included in any feasible combination. For example, the fixation system can further include a containment sleeve, wherein deployment is achieved by removing the containment sleeve. The containment sleeve can be generally tubular and the self-expanding element can radially expand from the constrained configuration to the deployed configuration. The bone screw can be associated with the self-expanding element after deployment and the bone screw can be thread-associated with the self-expanding element and/or at least one attachment feature. The at least one attachment feature can be located distal to the self-expanding element and the bone screw can be a pedicle screw for fixation to the spine.

Another implementation of the fixation system can include a bone screw associated with a self-expanding element. The bone screw can be deliverable into the boney structure with the self-expanding element radially constrained and wherein the self-expanding element can be configured to be subsequently deployed into a radially expanded configuration relative to the long axis of the bone screw. In some variations the bone screw can be thread-associated with the self-expanding element and/or at least one attachment feature after deployment into the radially expanded configuration.

In addition, another implementation of the fixation device can include a pedicle screw having a threaded shaft and a receiver element modularly associated with the threaded shaft via a tapered shaft and tapered bore providing an interference fit. The fixation device can further include a locking element configured to secure the interference fit. In some variations the locking element can include a compression screw, an interference pin or a screw and a relative position of rotation along the long axis of the threaded shaft is infinitely adjustable. The modular receiver element can be selected from a plurality of receiver elements having different lengths.

An implementation of the bone screw described herein includes a modular bone screw that has a bone screw having a threaded shaft, a connecting element, a plurality of modular spacers, and a receiver element. The receiver element can be configured to be associated with one of the plurality of modular spacers below and one of the plurality of modular spacers above the connecting element providing increased surface area contact between the spacers and the connecting element with final receiver element engagement. In addition, an axis of a channel can be generally defined by the plurality of modular spacers and can be between 89 degrees and 121 degrees relative to the long axis of the threaded shaft. Additionally, the modular bone screw can include a set screw associated with modular spacer positioned above the connecting element.

An implementation of a fixation system is also described herein that can include a pedicle screw having an elongated body with a proximal head and a distal shaft, the distal shaft including threads radially extending along an outer surface of the distal shaft for securing the distal shaft into the boney structure. In addition, the fixation system can include a receiver element having a pair of upper opposing projections with a first channel formed between the pair of upper opposing projections, the first channel being shaped to receive a part of a connecting rod, the receiver element further comprising a pair of lower opposing projections with a second channel formed between the pair of lower opposing projections, the second channel being shaped to mate with the proximal head of the pedicle screw. Additionally, the fixation system can include an expanding element having a proximal tubular portion, a distal tubular portion, and a plurality of flexible arms that extend between the proximal and distal tubular portions, the expanding element forming a tubular shape when a compressive force is applied along the plurality of flexible arms, the plurality of flexible arms being self-expanding and bowing radially outward when the compressive force is released such that the distal expanding element forms a radially expanded shape.

In some variations one or more of the following features can optionally be included in any feasible combination. For example, the fixation system can include a proximal end of the expanding element is more radially expanded compared to a distal end of the expanding element when the expanding element is in the radially expanded shape. The expanding element can be made out of one or more of a superelastic material, a shape-set material, and a Nitinol material. The fixation system can further include a distal coupler that couples to the distal tubular portion of the expanding element, the distal coupler having an inner channel that threadably couples to the distal shaft of the pedicle screw, the distal coupler allowing for reversibly threaded association of the expanding element with the distal shaft. The fixation system can further include a cylindrical containment sleeve that, when in a first position, applies the compressive force along the plurality of flexible arms, the containment sleeve being movable from the first position thereby releasing the compressive force along the plurality of flexible arms. The fixation system can further include an axle that extends through an aperture located along each of the lower opposing projections, the axle further extending through the proximal head of the pedicle screw for pivotally coupling the receiver element to the pedicle screw. The fixation system can further include a set screw having exterior threaded features that engage with interior threaded features along the pair of upper opposing projections for securing the set screw to the receiver element and capturing the part of the connecting rod in the first channel. The proximal head of the pedicle screw can include a distal stem that extends from the proximal head and is shaped to slidably mate with a bore extending a distance into the distal shaft, at least one of the proximal head and the receiver element being rotatable relative to the distal shaft and rotatable along a longitudinal axis of the distal shaft. The distal stem and the bore can have a tapered shape that form an interference fit when coupled together. The distal stem includes a first anti-rotation feature that engages a second anti-rotation feature along the bore for preventing rotation of the distal stem relative to the bore at least when the proximal head is secured to the distal shaft. The fixation system can further include a locking screw that secures the proximal head to the distal shaft thereby preventing rotation therebetween. The fixation system can further include one or more spacers configured to sit in the first channel at least one of below and above the part of the connecting rod extending along the first channel, the one or more spacers having a rod conforming section that is shaped to correspond to a surface geometry of the part of the connecting rod. The rod conforming section can include at least one of a vaulted geometry, a concave geometry, a surface texture, and an angled surface relative to a longitudinal axis of the distal shaft of the pedicle screw. The pedicle screw can be cannulated. The proximal head can include at least one of a scalloped shape and a splined surface configured to interact with the part of the rod extending through the first channel.

In another interrelated aspect of the current subject matter, a method can include engaging a distal end of a fixation system into a boney structure, the fixation system including a pedicle screw having an elongated body with a proximal head and a distal shaft, the distal shaft including threads radially extending along an outer surface of the distal shaft for securing the distal shaft into the boney structure. The fixation system can further include a receiver element having a pair of upper opposing projections with a first channel formed between the pair of upper opposing projections, the first channel being shaped to receive a part of a connecting rod, the receiver element further comprising a pair of lower opposing projections with a second channel formed between the pair of lower opposing projections, the second channel being shaped to mate with the proximal head of the pedicle screw. In addition, the fixation system can include an expanding element having a proximal tubular portion, a distal tubular portion, and a plurality of flexible arms that extend between the proximal and distal tubular portions, the expanding element forming a tubular shape when a compressive force is applied along the plurality of flexible arms, the plurality of flexible arms being self-expanding and bowing radially outward when the compressive force is released such that the distal expanding element forms a radially expanded shape. Additionally, the fixation system can include releasing the compressive force along the plurality of flexible arms thereby allowing at least one of the plurality of flexible arms to bow radially outward within the boney structure.

The method can further include receiving the part of the connecting rod within the first channel and advancing a set screw between the upper opposing projections to secure the part of the connecting rod within the first channel. Furthermore, the method can include rotatably moving the proximal head relative to the distal shaft and securing the orientation of the proximal head relative the distal shaft, the securing including threadably engaging a locking screw extending from the proximal head into the distal shaft.

In another interrelated aspect of the current subject matter, a method of treating a boney structure with a fixation device is described. The fixation device can include a bone screw and a self-expanding element configured to be associated with the bone screw, and the method can include deploying the bone screw in the boney structure and deploying the self-expanding element to be associated with the bone screw. In addition, the method can include optimizing resistance to displacement of the bone screw by manipulating the deployed self-expanding element. Furthermore, the manipulation can include pulling the fixation device along an axis of delivery of the bone screw and the bone screw can be associated with the self-expanding element after manipulation.

The details of one or more variations of the subject matter described herein are set forth in the accompanying drawings and the description below. Other features and advantages of the subject matter described herein will be apparent from the description and drawings, and from the claims

### BRIEF DESCRIPTION OF THE DRAWINGS

These and other aspects will now be described in detail with reference to the following drawings. Generally speaking the figures are not to scale in absolute terms or comparatively but are intended to be illustrative. Also, relative placement of features and elements may be modified for the purpose of illustrative clarity.

FIG. 1 illustrates an isometric view of an implementation of a pedicle screw assembly;

FIGS. 2A-2C illustrate exploded, partial views of the pedicle screw assembly of FIG. 1;

FIG. 3A illustrates a cross-sectional view of the pedicle screw assembly of FIG. 1;

FIG. 3B illustrates an exploded, partial view of the pedicle screw assembly of FIG. 1;

FIGS. 3C-3E illustrate various views of modular spacers for use with the pedicle screw assembly of FIG. 1;

FIGS. 4A-4C illustrate receiver elements having various lengths;

FIG. 5A illustrates a perspective view of an interrelated implementation of a pedicle screw assembly;

FIGS. 5B and 5C illustrate side views of the pedicle screw assembly of FIG. 5A;

FIG. 5D illustrates a cross-sectional view of the pedicle screw assembly of FIG. 5A;

FIG. 5E illustrates an exploded view of the pedicle screw assembly of FIG. 5A;

FIG. 6A illustrates an initial guide pin inserted within a vertebral body;

FIG. 6B illustrates a cannulated reamer advanced over the initial guide pin of FIG. 6A to create a channel along a pedicle axis;

FIG. 6C illustrates expansion of the channel of FIG. 6B using an impaction reamer;

FIG. 6D illustrates delivery of a self-expanding element through a containment sleeve;

FIG. 6E illustrates the self-expanding element coupled to a positioner and a guide pin;

FIG. 6F illustrates a cross-sectional view of the assembly of FIG. 6E taken along section A-A;

FIG. 6G illustrates expulsion of the self-expanding element from the containment sleeve;

FIG. 6H is a side view of the expanded element coupled to the positioner and the guide pin;

FIG. 6I illustrates a cross-sectional view of the assembly of FIG. 6H taken along section B-B;

FIG. 6J illustrates adjustment of the position of the self-expanding element against the pedicle root after delivery and expansion;

FIG. 6K illustrates association of a pedicle screw assembly with the expanded self-expanding element of FIG. 6J;

FIGS. 6L and 6M illustrate a side and a cross-sectional view of the pedicle screw shaft associated with the self-expanding element taken along section C-C.

It should be appreciated that the drawings herein are exemplary only and are not meant to be to scale.

### DETAILED DESCRIPTION

The present disclosure relates generally to orthopedic boney fixation systems and methods, and more particularly, to pedicle fixation systems and methods. The systems and methods described herein provide a fixation system that facilitates association between the pedicle screw receiver element and the connecting rod and enhanced pedicle screw fixation, particularly in reduced density bone and also in long segmental constructs. The systems described herein provide for removal and/or revision even when bone has grown within the architecture of the self-expanded component of the pedicle fixation system.

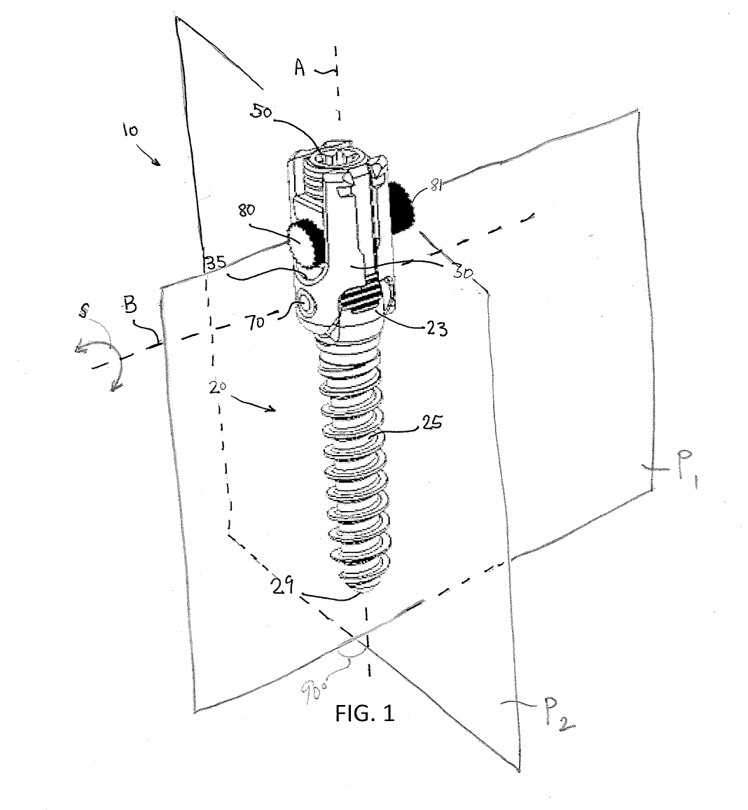

FIG. 1 shows an isometric view of an implementation of a pedicle screw assembly 10 having a pedicle screw 20 coupled to a receiver element 30 having a “U” shaped channel 35 within which a connecting rod 80 can reside. The pedicle screw assembly 10 can also include a set screw 50 configured to mate with the receiver element 30 to secure the connecting rod 80 within the channel 35. Receiver elements of known pedicle screw fixation systems can be in a monolithic, fixed relationship to the pedicle screw shaft (i.e. mono-axial pedicle screw) or a spherically-adjustable relationship with the pedicle screw shaft (i.e. poly-axial pedicle screw). While poly-axial pedicle screws can provide a measure of adjustability that makes association with connecting rods simpler, experimental studies have demonstrated that mono-axial pedicle screws with a monolithic design tolerate higher loads to failure, particularly rotational slippage of the poly-axial joint. As will be described in more detail below, the pedicle screw assemblies described herein provide a modularly adjustable screw to accommodate for various positions of a segmental fixation connecting rod relative to the pedicle screw shaft while preserving stability characteristics more commonly associated with the mono-axial design.

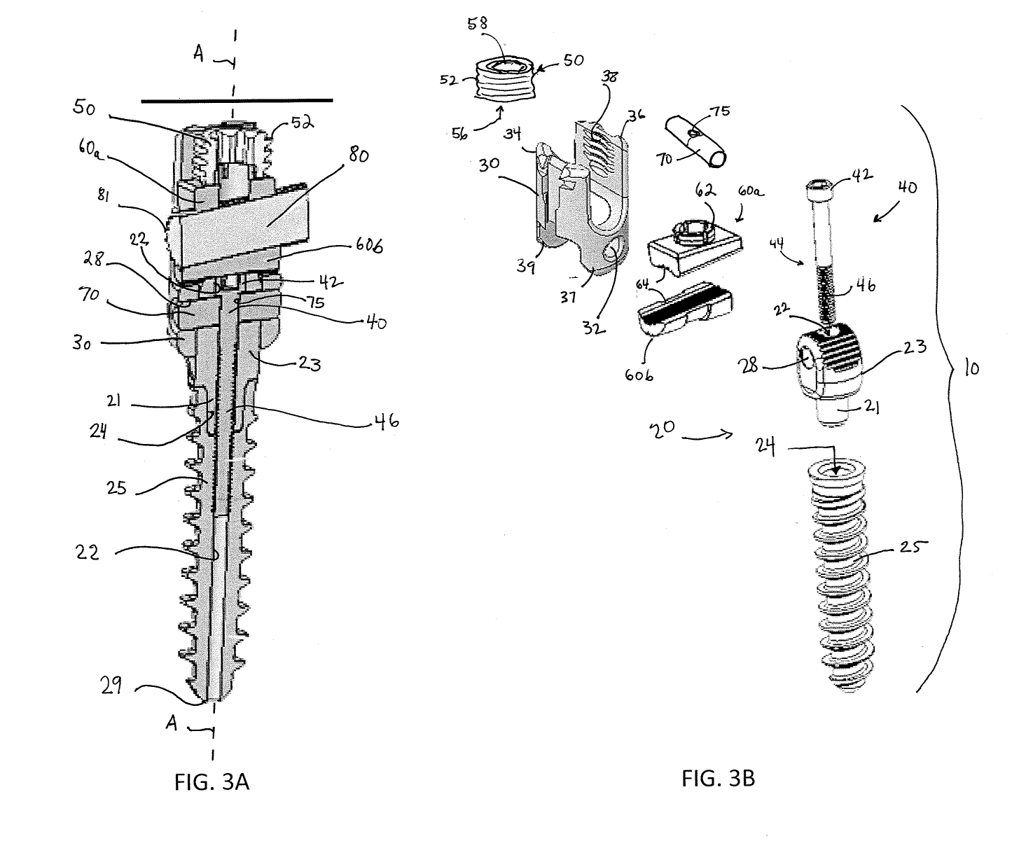

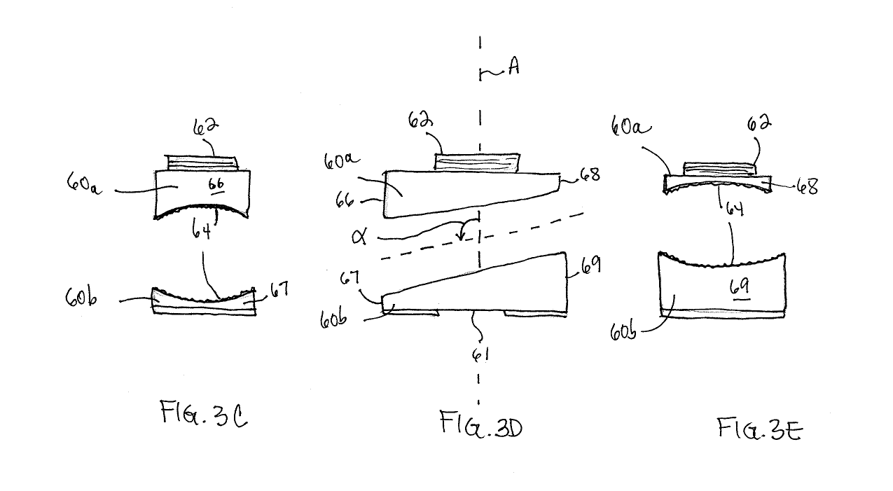

Again with respect to FIG. 1, the pedicle screw 20 can have a proximal head 23 configured to mate with the receiver element 30 and a distal shaft 25 configured to penetrate bone. The distal shaft 25 can be threaded and have a distal tip 29, which may have a chamfer to aid insertion into the pedicle boney process. In some implementations, the chamfer is about 60 degrees. As best shown in FIG. 3A, the pedicle screw 20 can include a cannulation 22 extending through at least a portion of an interior of the pedicle screw 20. In some implementations, the cannulation 22 extends through a proximal end region of the pedicle screw 20, for example through the proximal head 23. In some implementations, the cannulation 22 extends from a proximal end of the screw 20 clear through to the distal tip 29 of the pedicle screw 20 such that the entire length of the screw 20 along its central axis A is cannulated.

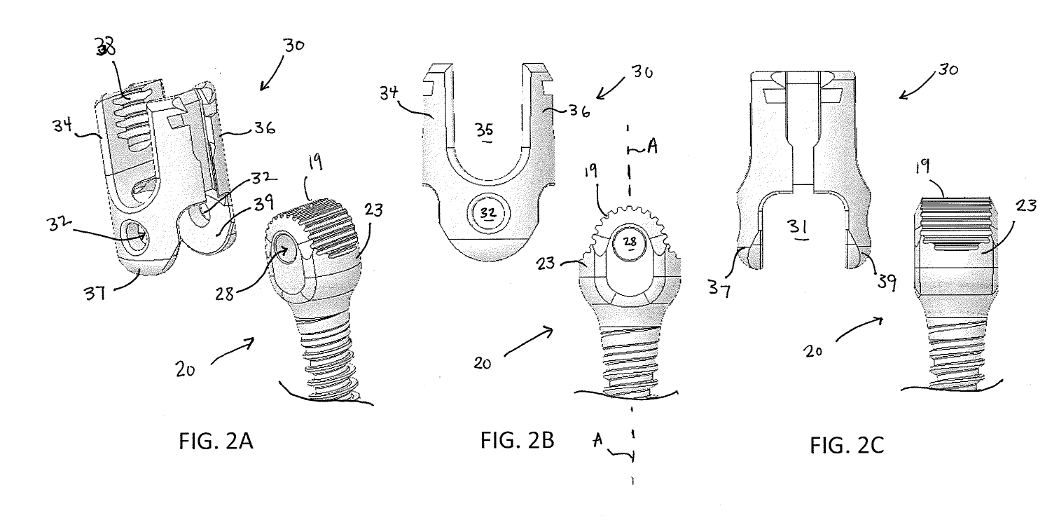

Now with respect to FIGS. 2A-2C, the proximal head 23 of the pedicle screw 20 can have a bore 28 extending through it that is configured to receive an axel 70 (see FIG. 1). The axel 70 can couple the proximal head 23 of the pedicle screw 20 within a region of the receiver element 30. As mentioned above, the proximal receiver element 30 can have a “U” shaped channel 35 within which the connecting rod 80 can reside. The channel 35 can be formed by opposing upper projections 34, 36 adapted to receive the connecting rod 80 therethrough. The receiving element 30 can also include opposing lower projections 37, 39 forming a second channel 31. The opposing lower projections 37, 39 can have opposed apertures 32 extending therethrough forming a bore configured to receive the axel 70. The proximal head 23 of the pedicle screw 20 can be positioned within the second channel 31 between the opposing lower projections 37, 39 of the receiving element 30 such that the apertures 32 align with the bore 28 of the proximal head 23 of the pedicle screw 20. The axel 70 can extend through the apertures 32 and the bore 28 engaging the proximal head 23 of the pedicle screw 20 within the second channel 31 of the receiving element 30 while permitting the pedicle screw 20 to rotate freely about the axel 70 via its bore 28. This allows the receiver element 30 to be swiveled relative to the longitudinal axis A of the pedicle screw 20 around the axis B along arrow S (see FIG. 1). The U-shaped channel 35 can have a first plane P₁ and the second channel 31 can have a second plane P₂ and the first plane P₁ and the second plane P₂ can extend perpendicular to one another. Thus, the plane of articulation of the receiver element 30 around the axel 70 and axis B is likewise perpendicular to the first plane P₁ of the U-shaped channel 35. The receiver element 30 is prevented from rotating along the long axis the connecting rod 80. In other words, because the axis of the channel 35, as defined by the general course of the longitudinal axis of the rod 80 within the channel 35, is oriented at 90 degrees relative to the pedicle screw shaft axis, the pedicle screw assembly 10 can allow for articulation of the pedicle screw 20 between the opposing lower projections 37, 39 of the receiver element 30, but prevents articulation of the pedicle screw 20 along the long axis of the connecting rod 80. It should be appreciated that the orientation of the first channel 35 and the second channel 31 can vary and need not be limited to being oriented at 90 degrees relative to one another.

As described above, the receiver element 30 can be oriented various degrees offset from the longitudinal axis A of the pedicle screw 20 along a single plane. The proximal head 23 of the pedicle screw 20 may have a surface geometry such that it can more effectively mate with the rod 80 and mate according to various pre-determined angles relative to the rod 80 positioned within the receiver element 30. For example, the proximal head 23 of the pedicle screw 20 can have a scalloped shape as described in U.S. Pat. No. 7,780,706, which is incorporated herein by reference in its entirety. The proximal head 23 of the pedicle screw 20 can also have a splined surface 19, for example, as shown in FIGS. 2A-2C, that can mate with corresponding splined surface 81 of the rod 80 (see FIG. 1). It should be appreciated, however, that the rod 80 need not mate with the proximal head 23 of the pedicle screw 20 and instead the pedicle screw assembly 10 can include an intervening element between the rod 80 and the pedicle screw head 23 as will be described in more detail below.

In some implementations, the pedicle screw assembly 10 can have an additional level of rotational adjustability about the axis of the connecting rod channel 35 (see FIGS. 3A-3B). As best shown in FIG. 3B, the proximal head 23 of the pedicle screw 20 can be coupled to the receiver element 30 as described above. In this implementation, the proximal head 23 forms a base of the receiver element 30 and separate from the distal threaded shaft 25 of the pedicle screw 20. This modular association of the receiver element 30 to the pedicle screw threaded shaft 25 can allow for the relative position of rotation of the receiver element 30 around the central axis A that is infinitely adjustable. The relative position of the receiver element 30 to the threaded shaft 25 can be adjusted and optimized providing the assembly 10 with an additional level of adjustability beyond the mono-planar rotation described above.

Still with respect to FIGS. 3A-3B, the proximal head 23 can include a distal stem 21 that is configured to couple to a proximal aspect of the threaded shaft 25. In some implementations, the stem 21 can be sized to fit within a bore 24 extending a distance into the proximal aspect of the pedicle screw shaft 25. The stem 21 and bore 24 can each be tapered such that they wedge together by interference fit upon advancement of the stem 21 into the bore 24. The interference fit between the tapered stem 21 and tapered bore 24 can be secured with a compression screw, interference pin, or other element. As described above, the pedicle screw 20 can include a cannulation 22 extending through at least a portion of the screw 20. The cannulation 22 can extend from a first end of the proximal head 23 through the stem 21 and into the threaded shaft 25 coaxial with the central axis A and the bore 24. A locking screw 40 can insert through the cannulation 22 in the proximal head 23 into at least a region of the cannulation 22 within the threaded shaft 25. In some implementations, the locking screw 40 can have a proximal head 42 and a distal shaft 44 having a threaded region 46. The threaded region 46 of the locking screw 40 can engage corresponding threads in at least a portion of the cannulation 22 locking the stem 21 within the bore 24 to securely associate the receiver element 30 with the threaded shaft 25 via the head 23. As described above, the axel 70 can extend through bore 28 of the proximal head 23 and apertures 32 of the lower projections 37, 39 to maintain the engagement between the head 23 and the receiver element 30. Cannulation 22 and bore 28 can intersect near the central axis A. As such, axel 70 can include an aperture 75 configured to receive the locking screw 40 when positioned through cannulation 22 (best shown in FIG. 3A).

Upon optimization of the relative rotation between the receiver element 30 and the shaft 25 of the pedicle screw 20, the association between the stem 21 positioned inside the bore 24 can be locked by advancing the locking screw 40 into cannulation 22 preventing any further rotation. In some implementations, the stem 21 and the bore 24 can mate using a locking taper, such as a Morse taper geometry, such that in the final locked position the two taper lock geometries are closely approximated to secure the proximal head 23 to the threaded shaft 25. Rotation of the locking screw 40 and additional threaded engagement between the threaded region 46 of the locking screw 40 and the corresponding threads in the cannulation 22 of the shaft 25 pull the two parts into tighter association until the tapered regions wedge together and bind to each other. The stem 21 can be discontinuous or axially slotted such that axial, distal advancement of the locking screw 40 or locking pin (not shown) within the proximal head 23 can splay, displace or expand at least a portion of the stem 21 to further wedge the parts to each other and securely lock the proximal head 23 relative to the pedicle screw shaft 25.

The stem 21 can include at least one anti-rotational feature that keys or interlocks with a corresponding anti-rotational feature on at least a part of the mating surface geometry of the bore 24 in the shaft 25 when the proximal head 23 is secured to the shaft 25. It should be appreciated that any of a variety of projecting features engaged within corresponding longitudinal recesses can be incorporated to prevent rotation of the stem 21 with respect to the bore 24.

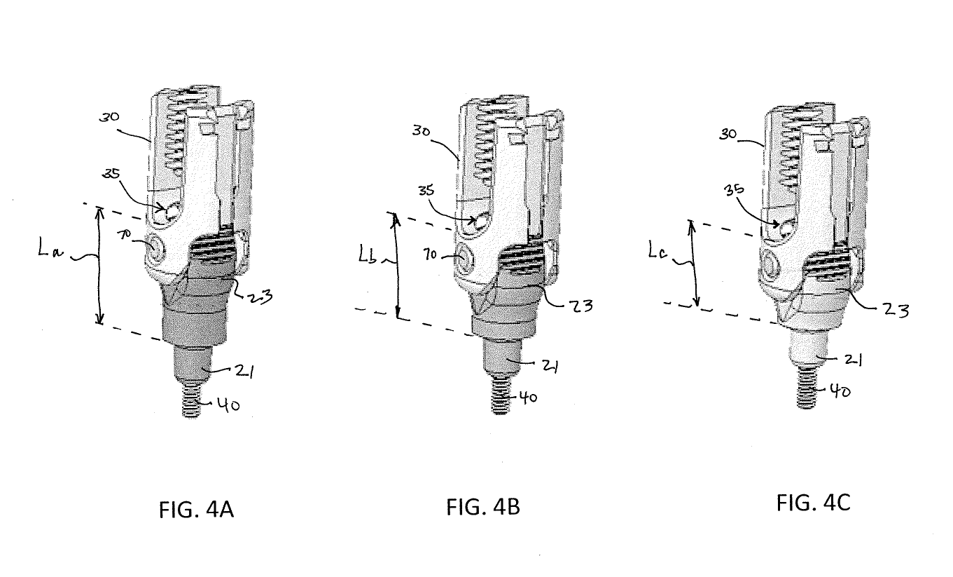

The length of the proximal head 23, and thus the height of the receiver element 30 extending above the threaded shaft 25 of the pedicle screw, can vary. This provides another dimension of adjustability for the receiver element 30 in addition to the angulation and rotational adjustments described above. For example, FIGS. 4A-4C show differing lengths of a region of the proximal head 23 located proximal to the stem 21 and distal to the channel 35. FIG. 4A illustrates a first length La providing a greater height to the receiver element 30 compared to the lengths Lb and Lc shown in FIGS. 4B or 4C, respectively.

Again with respect to FIGS. 3A-3B, the pedicle screw assembly 10 can also include a set screw 50 configured to mate with the receiver element 30 to secure the connecting rod 80 within the channel 35. After the pedicle screw 20 is advanced into a pedicle channel of the vertebra, the connecting rod 80 may be inserted into the receiver element 30. A desired rotational orientation between the pedicle screw 20 and the receiver element 30 can be achieved (i.e. around the axel 70 axis B and/or around the central axis A), the set screw 50 may be inserted to compress the rod 80 against the proximal head 23 of the screw 20 (or against an intervening element as described in more detail below) to maintain the desired orientation. The opposing upper projections 34, 36 can each include a thread 38, such as a female thread, on their inner surfaces and the set screw 50 can include a thread 52, such as a male thread, on its external surface that is dimensioned to mate with threads 38 and thus, engage the projections 34, 36. When the set screw 50 is inserted into the opposing upper projections 34, 36, and rotated around the longitudinal axis A of the assembly 10 the mating thread patterns 38, 52 can prevent or limit splaying of the projections 34, 36 away from the longitudinal axis of the screw 20 as the set screw 50 is advanced further into the channel 35. The set screw 50 can include a recessed key 58 configured to receive a tool to rotate the set screw 50.

As mentioned above, the assembly 10 can further include one or more spacers 60 configured to be positioned within the receiver element channel 35 and positioned either above or below, or both above and below the connecting rod 80. The modular spacers 60 can improve the surface apposition of the connecting rod 80 to the receiver element 30 by providing increased surface areas of contact. In some implementations, the assembly 10 includes an upper spacer 60a and a lower spacer 60b. The upper spacer 60a can be integrated within, such as rotatably, or coupled to or extending through at least a portion of the set screw 50. For example, the upper spacer 60a can include a head 62 dimensioned to be inserted into the receiving chamber 56 of the set screw 50 such that the head 62 and the set screw 50 do not become disengaged while still enabling the set screw 50 to be rotated independently of the upper spacer 60a. The spacers 60 can include a rod conforming section 64 shaped to correspond to an overall surface geometry of the rod 80 and to sandwich the connecting rod 80 (if there are two spacers) and provide an intimate surface contact fit, for example, between the connecting rod 80 and the spacers 60 upon advancement of the set screw 50. The rod conforming section 64 can include reduced ends that can cause the spacers 60 to bend a rod 80 when the set screw 50 compresses the spacer 60 against the rod 80. In some implementations, the spacer 60 can help the rod 80 meet the natural lordosis of one or more vertebrae that the rod 80 may be coupled thereto via the pedicle screw assembly 10. The rod conforming section 64 can have a vaulted, concave geometry configured to match the outer convex geometry of the rod 80. The surface of the conforming sections 64 can include surface textures or features such as a splined surface to further engage with a corresponding surface texture or features on the rod 80.

The spacers 60 can form an angle relative to the longitudinal axis A. As best shown in FIGS. 3A (and also FIGS. 3C-3E), the connecting rod 80 is shown in cross-section secured between upper and lower angled receiver channel spacers 60a, 60b. The locking screw 40 that locks the stem 21 of the proximal head 23 to the tapered bore 24 of the threaded shaft 25 is contacted by the lower surface 61 of the lower spacer 60b whereas the upper spacer 60b can serve as a washer between the set screw 50 and the connecting rod 80. FIG. 3C illustrates an end view, FIG. 3D illustrates a side view, and FIG. 3E illustrates an end view of an implementation of the spacers 60a, 60b. The upper spacer 60a can have a first end 66 that is wider than the second end 68 and the lower spacer 60a can have a first end 67 that is narrower than the second end 69 such that upon pairing the upper and lower spacers 60a, 60b together within the receiver element 30 a channel for the connecting rod between the inner facing, conforming surfaces 64 forms an angle a relative to the longitudinal axis A of the pedicle screw shaft. In some implementations, the angle a can be 90 degrees such that the conforming surfaces 64 of each of the upper and lower spacers 60a, 60b are parallel to one another and to the axis of the connecting rod channel. Alternatively, the angle a can be other than 90 degrees relative to the longitudinal axis A. In some implementations, the angle a can be between about 89 degrees to about 121 degrees relative to the longitudinal axis A.

The devices, systems, assemblies and methods described herein can incorporate any of a variety of features described herein and elements or features of one implementation of a device and system described herein can be incorporated alternatively or in combination with elements or features of another implementation of a device and system described herein as well as various devices and features described in U.S. Pat. No. 7,780,706; U.S. Pat. No. 8,460,308; U.S. Publication No. 20090299412; and U.S. Pat. No. 7,951,152, which are each incorporated by reference herein in their entireties. For the sake of brevity, explicit descriptions of each of the combinations may be omitted although the various combinations are to be considered herein.

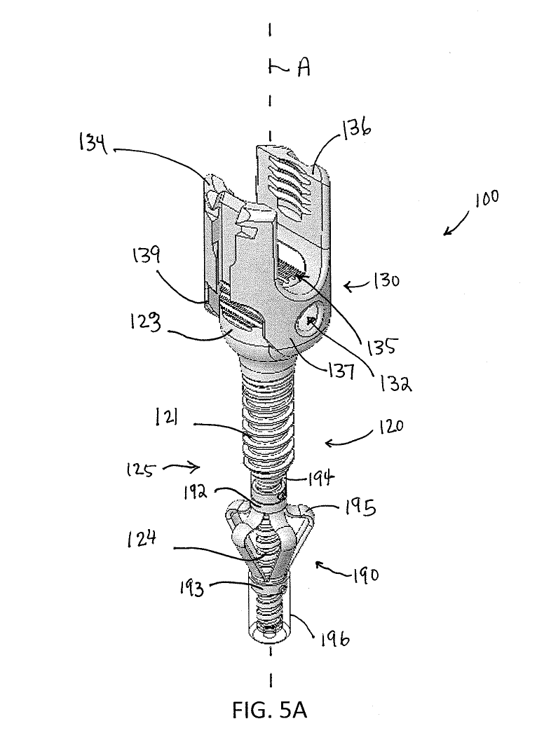

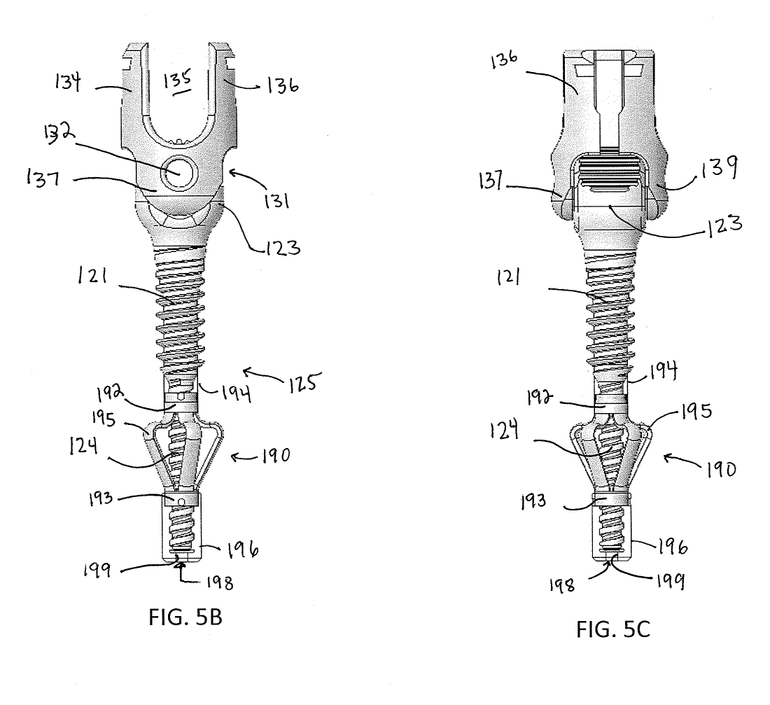

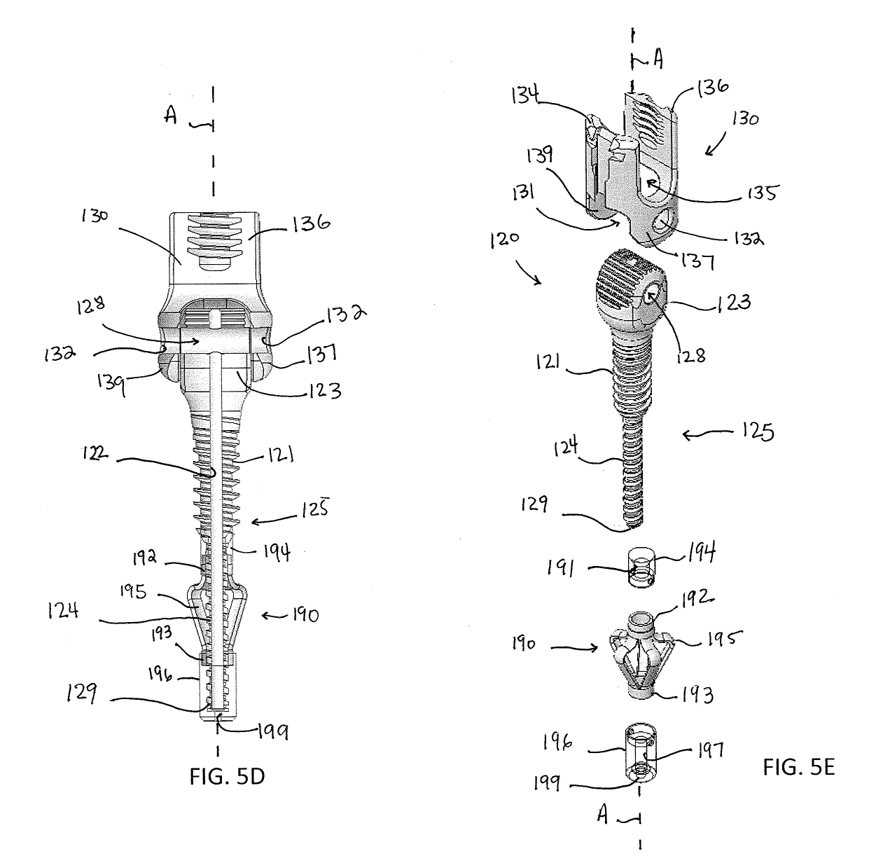

FIGS. 5A-5E illustrate an interrelated implementation of a pedicle screw assembly 100 having a pedicle screw 120 coupled to a distal expanding element 190 configured to optimize the enhanced resistance of the pedicle screw 120 to withdrawal failure of pull-out along the axis of insertion, which will be described in more detail below. The pedicle screw 120 of the pedicle screw assembly 100 can be coupled to a receiver element 130 having a “U” shaped channel 135 within which a connecting rod (not shown) can reside and a set screw (not shown) configured to mate with the receiver element 130 to secure the connecting rod within the channel 135. The pedicle screw 120 can have a proximal head 123 configured to mate with the receiver element 130 and a distal shaft 125 configured to penetrate bone. The distal shaft 125 can include a proximal region 121 and a distal, reduced diameter region 124. At least a portion of the proximal and distal regions 121, 124 can be threaded. The pedicle screw 120 can include a cannulation 122 extending through at least a portion of an interior of the pedicle screw 120 (shown in FIG. 5D). In some implementations, the cannulation 122 extends through the proximal head 123 of the pedicle screw 120 clear through to a distal tip 129 such that the entire length of the screw 120 is cannulated along its central axis A.

As mentioned above, the proximal receiver element 130 can have a “U” shaped channel 135 within which the connecting rod can reside. The channel 135 can be formed by opposing upper projections 134, 136 adapted to receive the connecting rod therethrough. The receiving element 130 can also include opposing lower projections 137, 139 forming a second channel 131. The channel 135 can have a first plane and the second channel 131 can have a second plane such that the first plane and the second plane are perpendicular to one another. The opposing lower projections 137, 139 can have opposed apertures 132 extending therethrough forming a bore configured to receive an axel (not shown). As described herein, the proximal head 123 of the pedicle screw 120 can have a bore 128 intersecting the cannulation 122 that is configured to receive the axel. The axel can couple the proximal head 123 of the pedicle screw 120 within a region of the receiver element 130. The proximal head 123 of the pedicle screw 120 can be positioned within the second channel 131 between the opposing lower projections 137, 139 of the receiving element 130 such that the apertures 132 align with the bore 128 of the proximal head 123 of the pedicle screw 120. The axel can extend through the apertures 132 and the bore 128 engaging the proximal head 123 of the pedicle screw 120 within the second channel 131 of the receiving element 130 while permitting the pedicle screw 120 to rotate freely about the axel via its bore 128. This allows the receiver element 130 to be swiveled relative to the longitudinal axis A of the pedicle screw 120 as described in more detail above. The receiver element 130 is shown as being allowed to articulate along a first plane and prevented from articulating along a second perpendicular plane. It should be appreciated that in some implementations, the receiver element 130 can articulate along a second plane and be prevented from articulating along the first perpendicular plane. The receiver element 130 can articulate along a different plane that what it shown. Further, in other implementations the receiver element 130 does not articulate and is fixed.

The pedicle screw 120 can include a threaded shaft 125 having a proximal region 121 and a distal, reduced diameter region 124 (see, for example, FIG. 5E). It should be appreciated that the proximal region 121 and also the distal region 124 may be only partially threaded. The radially expanding element 190 can be a generally tubular element configured to receive the reduced diameter region 124 of the pedicle screw shaft 125 within its interior volume when the pedicle screw 120 is implanted. The expanding element 190 can include a proximal tubular portion 192, a distal tubular portion 193 and a plurality of flexible arms 195 extending between the proximal and distal tubular portions 192, 193. The plurality of flexible arms 195 can be self-expanding such that upon release of a compressive force they each “relax” into a particular shape set configuration. For example, the flexible arms 195 can bow radially outward such that the expanding element 190 assumes a volumetrically-enlarged geometry. The geometry attained can vary. In some implementations, the enlarged geometry can be generally oblong such that a proximal end of the expanding element 190 (e.g. an end nearest the root of the pedicle within which it is implanted) is more radially expanded compared to the distal end of the expanding element 190. The volumetrically-enlarged geometry of the expanding element 190 is generally characterized by axial foreshortened as the outer dimension radially expands. The expanding element 190 can include one, two, three, four, five or more flexible arms 195 that can be disposed symmetrically or asymmetrically around the central axis A.

At least a portion of the expanding element 190, such as the flexible arms 195 can be formed of a superelastic, shape-set material or combination of materials including Nitinol. Suitable materials or combinations of materials for the preparation of the various components of the devices disclosed herein can vary. It should be appreciated that other suitable materials are considered for forming certain components for the devices described herein. The surface geometry of the expanding element 190 can be generally discontinuous such that the expanded element 190 has an open architecture following deployment. The width of the flexible arms 195 as well as the width between the flexible arms 195 can vary as can the thickness of the arms 195. The flexible arms 195 can be made thicker or thinner to achieve a particular strength, for example, to compress cancellous bone upon expansion and/or to prevent collapse and subsequent pull-out through the insertion axis. The flexible arms 195 can be flattened and have a wider dimension or the flexible arms 195 can be thinner and more rounded like a wire. The wall thickness and width of the flexible arms 195 can be uniform or non-uniform.

As best shown in FIGS. 5D and 5E, the proximal tubular portion 192 can be coupled to a proximal coupler 194 and the distal tubular portion 193 can be coupled to a distal coupler 196. For example, the distal end of the proximal coupler 194 can be coupled to the proximal tubular portion 192 of the expanding element 190 and the proximal end of the distal coupler 196 can be coupled to the distal tubular portion 193. The proximal coupler 194 has an inner channel 191 extending between a proximal end to a distal end of the proximal coupler 194. The distal coupler 196 also has an inner channel 197 extending from a proximal end towards a distal end of the distal coupler 196. The inner channels 191, 197 of the couplers 194, 196 can be threaded such that the couplers 194, 196 can mate with a corresponding threaded surface, for example, of the distal region 124 of the pedicle screw 120 or another threaded element. The inner channel 197 of the distal coupler 196 can have a distal opening 198 allowing for a guide pin or similar element to extend from the inner channel 197 through the distal opening 199. The inner channel 197 of the distal coupler 196 can additionally include a reduced diameter distal section 199 that is configured to mate with threads of a similarly reduced diameter guide pin, as will be described in more detail below. In some implementations, the expanding element 190 includes a distal coupler 196 and no proximal coupler 194 such that only the internal thread form of the distal coupler 196 engages the distal region 124 of the pedicle screw 120.

The couplers 194, 196 allow for reversibly threaded association of the self-expanding element 190 with the distal region 124 of the pedicle screw 120. When the expanding element 190 is associated with the pedicle screw 120, the pedicle screw 120 extends through the inner channels 191, 197 of the proximal and distal coupler 196 such that the proximal tubular portion 192 (and the proximal coupler 194) encircles a proximal portion of the reduced diameter region 124 and the distal tubular portion 193 (and distal coupler 196) encircles a distal portion of the reduced diameter region 124. The threads of the reduced diameter region 124 engage the corresponding threaded surfaces of the proximal coupler 194 and the distal coupler 196. In some implementations, a proximal portion of distal region 124 is non-threaded and has the diameter of the major diameter of the threaded distal segment that engages only coupler 196.

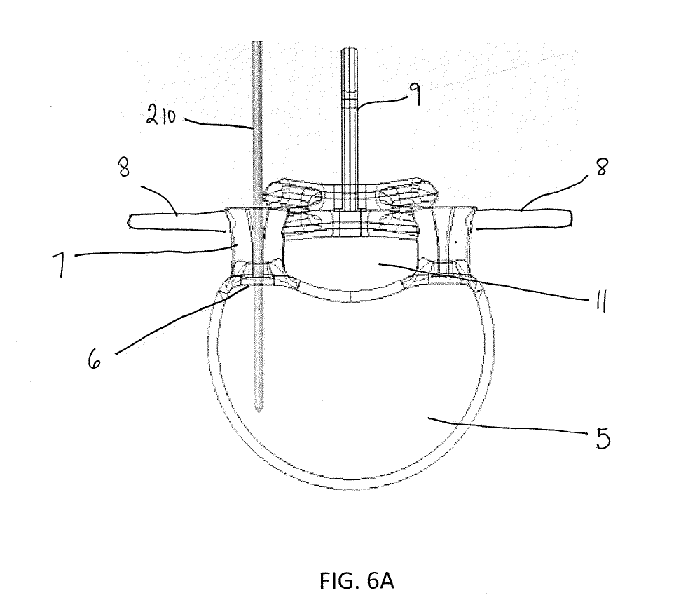

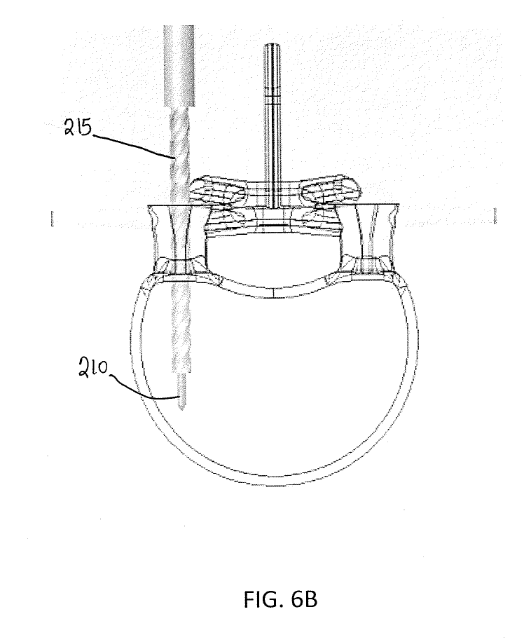

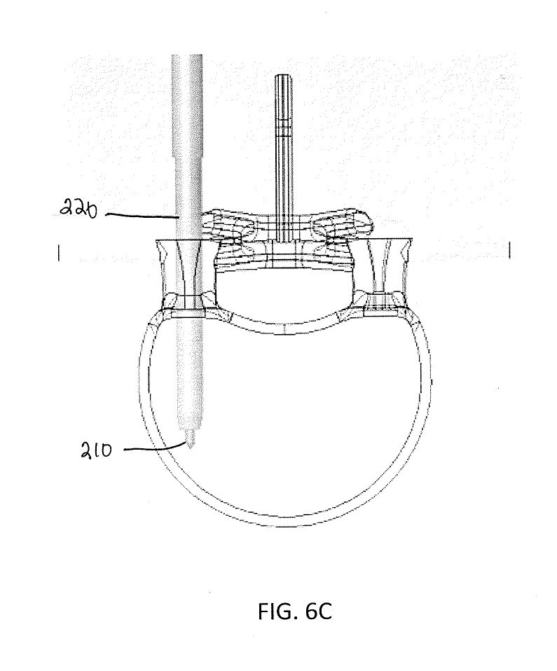

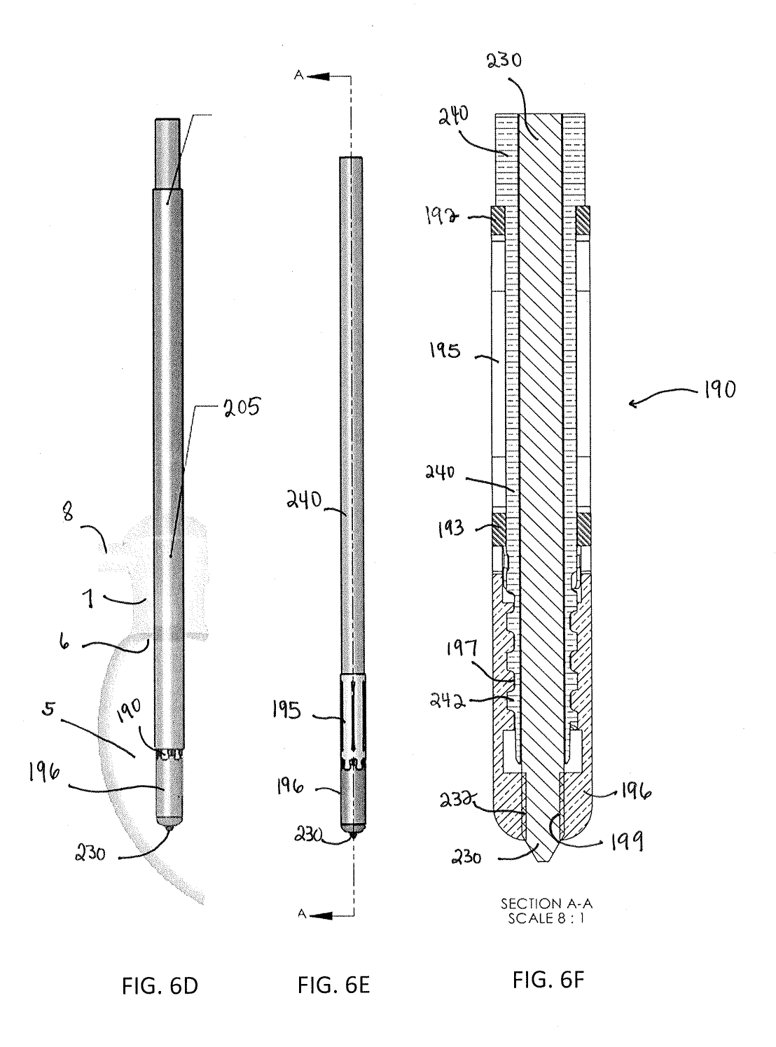

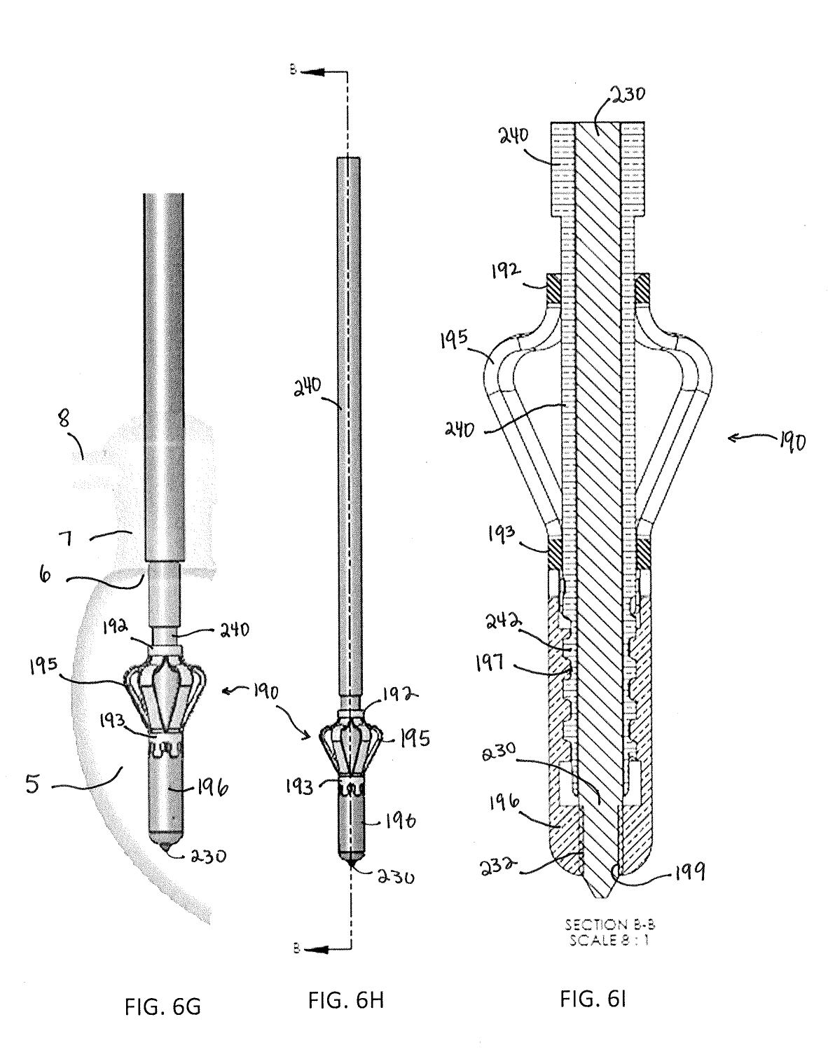

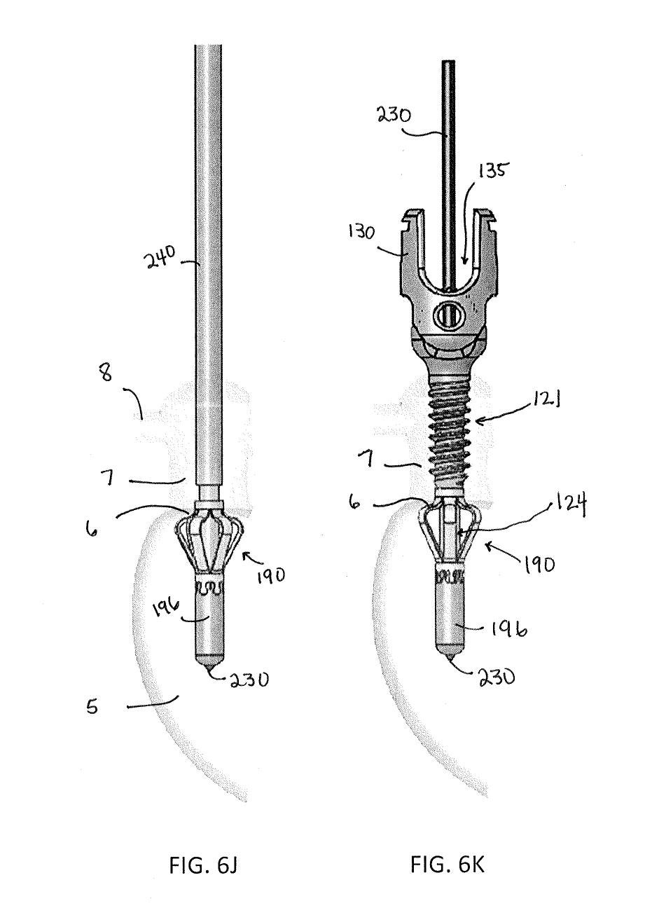

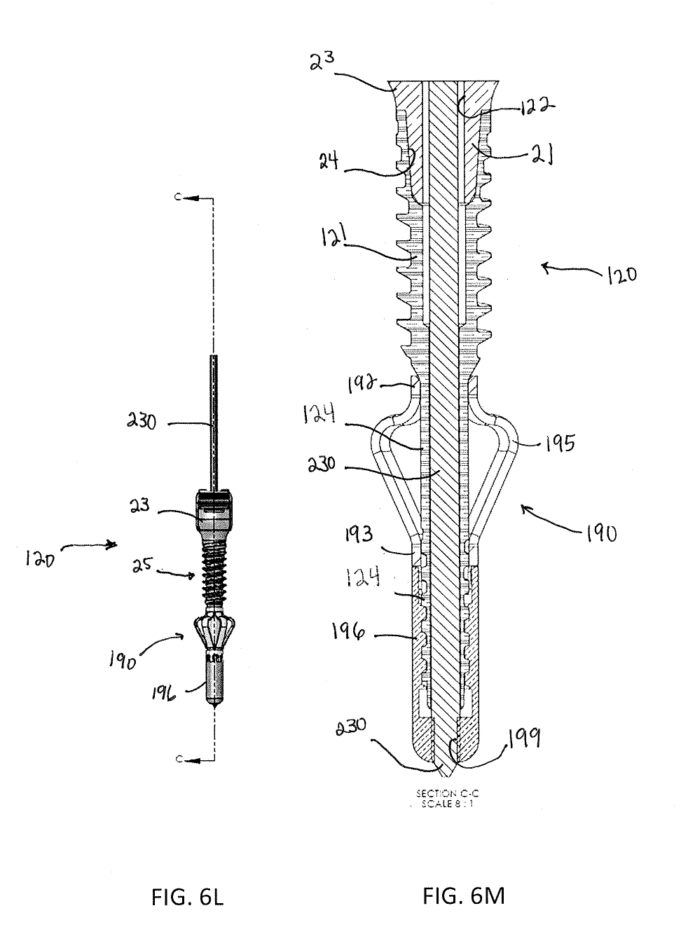

As will be described in more detail below, prior to insertion within the bone, the expanding element 190 can be maintained in a reduced dimension within a containment sleeve 205 (see FIGS. 6C and 6D). The containment sleeve 205 can be a hollow, cylindrical sheath that provides circumferential, inward force on the external surface of the expanding element 190 such that it is retained in a low profile, collapsed state during insertion. Withdrawal of the containment sleeve 205 in the proximal direction frees the expanding element 190 to relax into its expanded state. Although the expanding element 190 is generally self-expanding upon removal of the compressive forces of the containment sleeve 205, it should be appreciated that active expansion techniques or mechanisms can be applied to supplement or replace the self-expanding capabilities of the element 190.

The various devices and assemblies described herein can be implanted according to a variety of surgical methods known in the art. Depending upon the features and components of the assemblies, they can be implanted using various techniques or using various implements. FIGS. 6A-6D, 6G, 6J, and 6K show a step-wise implementation of a delivery method and a process for employing the assemblies described herein within a vertebra having a vertebral body 5, transverse processes 8, a posterior spinous process 9, a central canal 11, and pedicles 7 joined to the vertebral body 5 by a pedicle root 6. An initial guide pin 210 can be securely placed in the pedicle 7 along a pedicle axis and into the vertebral body 5 prior to initial reaming through the pedicle 7 (see FIG. 6A). A cannulated reamer 215 advanced over the initial guide pin 210 that can be used to create an initial channel extending coaxial to the pedicle axis (see FIG. 6B). The channel can be expanded with the aid of a distally tapered, radial impaction reamer or dilator 220 (see FIG. 6C). The dilator 220 can be advanced over the initial guide pin 210 and used to radially displace and compact cancellous bone along the bored pedicle axis within the channel away from the axis of insertion. Compacting the bone can provide for increased bone density and improved pedicle screw fixation, for example, in the case of osteoporotic bone.

The initial guide pin 210 can be removed and the self-expanding element 190 can be delivered in a constrained configuration through a thin walled containment sleeve 205 using a positioner 240 and a guide pin 230 into the channel of the pedicle 7 (see FIGS. 6D, 6E and 6F). All three of the self-expanding element 190, the guide pin 230, and the positioner 240 can be delivered together as a unit through the containment sleeve 205. The positioner 240 can be a cannulated element having external threads 242 configured to engage the internal threads of the inner channel 197 of the distal coupler 196. The guide pin 230 can have a smaller diameter than the initial guide pin 210, now removed, and has external threads 232 configured to engage internal threads of the reduced diameter distal section 199 of the distal coupler 196. The proximal coupler 194 can be, but is not necessarily, thread engaged with the threads of the guide pin 230 and/or external threads of the positioner 240. During delivery, the thin-walled containment sleeve 205 can cover a proximal aspect of the self-expanding element 190 and provide circumferential, inward force on the external surface of the expanding element 190 such that it is retained in a low profile, collapsed state during insertion through the channel. A distal edge of the containment sleeve 205 can be brought sufficiently anteriorly (i.e. distally) within the pedicle 7 to permit expulsion of the self-expanding element 190 from the containment sleeve 205 within the vertebral body 5 (see FIG. 6G). The containment sleeve 205 can be withdrawn proximally from covering the self-expanding element 190. Further, the positioner 240 and/or the reduced diameter guide pin 230 can provide the longitudinal displacing force to urge the self-expanding element 190 distal to the containment sleeve 205 such that the positioner 240 and/or guide pin 230 in combination with the containment sleeve 205 can be used in a push-pull manner to release the self-expanding element 190 from the containment sleeve 205.

Upon freeing the self-expanding element 190 from the circumferential force provided by the containment sleeve 205, the element 190 can spontaneously expand within the vertebral body 5 compacting adjacent soft cancellous bone (FIG. 6G). Following delivery and expansion of the self-expanding element 190 within the bone, active traction in a push-pull manner can be applied on the positioner 240 and the guide pin 230 to provide further expansion of the self-expanding element 190 ensuring it achieves full expansion. Further, the location of the self-expanding element 190 following delivery and expansion can be adjusted relative to the pedicle root 6 or axis of insertion and is not determined by the positioned of the pedicle screw 120. For example, one or both of the positioner 240 and the guide pin 230 can be used to manipulate or adjust the position of the expanded self-expanding element 190, for example, by pulling in a proximal direction (i.e. posteriorly). This can compress or compact the cancellous bone posterior to the self-expanding element 190 prior to association with the pedicle screw 120 to further enhance resistance to pull-out of the pedicle screw 120 once coupled to the distal coupler 196. Pulling in a proximal direction (i.e. posteriorly) can also position the self-expanding element 190 such that it abuts or is positioned immediately adjacent to the pedicle root 6 of the pedicle 7, which can optimize the enhanced resistance of an associated pedicle screw 120 to withdrawal failure or pull-out along the axis of insertion (see FIG. 6J).

Following optimization of the position of the expanded element 190, the positioner 240 can be unthreaded from the distal threaded coupler 196 (and optionally also the proximal threaded coupler 194) of the self-expanding element 190 and removed along with the confinement sleeve 205 leaving the centrally located and smaller diameter threaded guide pin 230 attached to the reduced diameter distal section 199 of the distal coupler 196. In some implementations, un-polymerized bone cement (e.g. methyl methacrylate) can be introduced into the vertebral body 5 prior to introducing the self-expanding element 190 and the cannulated and threaded self-expanding positioner 240 can be removed after the bone cement has polymerized. This can be done to further the pull-out strength of the pedicle screw assembly.

A pedicle screw 120 can be advanced over the guide pin 230 via the cannulation 122 of the pedicle screw 120 and threaded into the distal coupler 196 of the self-expanding element 190 (see FIGS. 6K, 6L, and 6M). The pedicle screw 120 can be simultaneously threaded into the radially expanded self-expanding element 190 as well as the pedicle 7. For example, the threads of the larger diameter proximal region 121 can have the same pitch as the threads of the reduced diameter distal region 124 such that upon rotation of the pedicle screw 120 the screw simultaneously threads into the pedicle 7 and the distal coupler 196 of the self-expanding element 190. The proximal region 121 can engage the pedicle 7, in particular, the isthmus of the pedicle 7, and the reduced-diameter, distal threaded region 124 of the pedicle screw 120 can thread engage distal coupler 196 or alternatively both the proximal and distal coupling features 194, 196 of the self-expanding element 190. The self-expanding element 190 is reversibly coupled to the pedicle screw 120 that despite the open architecture of the expanding element 190 allows for subsequent disassociation, removal, revision or replacement of the pedicle screw 120. For example, the pedicle screw 120 can be reversibly threaded from the distal coupler 196 of the expanding element 190 even upon ingrowth of soft tissue of bone within the expanded architecture of the flexible arms 195.

The guide pin 230 can be unthread from the distal coupler 196 and removed. A connecting rod can be inserted within the channel 135 of the receiver element 130 and fixed in place with the set screw as described elsewhere herein. It should be appreciated that the pedicle screw assembly 100 can incorporate any of a variety of other features described herein, for example, the modular receiver element described above that can include a stem 21 configured to be inserted within bore 24 such that the receiver element can be rotationally adjusted around the longitudinal axis A of the assembly. Similarly, the pedicle screw assembly 100 can include one or more spacers to adjust the position of the connecting rod relative to the pedicle screw shaft 125.

The devices, systems, assemblies and methods described herein can incorporate any of a variety of features described herein and that elements or features of one implementation of a device and system described herein can be incorporated alternatively or in combination with elements or features of another implementation of a device and system described herein as well as various devices and features described in U.S. Pat. No. 7,780,706; U.S. Pat. No. 8,460,308; U.S. Publication No. 20090299412; and U.S. Pat. No. 7,951,152, which are each incorporated by reference herein in their entireties. For the sake of brevity, explicit descriptions of each of those combinations may be omitted although the various combinations are to be considered herein. For example, the pedicle screw assemblies described herein can incorporate a receiver element that can be rotationally adjustable along a single plane as described herein. The receiver element can additionally or alternatively be adjustable around a central axis A as described herein. The receiver element can additionally or alternatively have varying height as described herein. The pedicle screw assembly can additionally or alternatively incorporate one or more fixed angle spacers as described herein. The pedicle screw assembly can additionally or alternatively incorporate a self-expanding element as described herein. Additionally, the devices and systems described herein can be positioned in the body and need not be implanted specifically as shown in the figures or as described herein. The various devices can be implanted, positioned and adjusted etc. according to a variety of different methods and using a variety of different devices and systems. Provided are some representative descriptions of how the various devices may be implanted and positioned, however, for the sake of brevity explicit descriptions of each method with respect to each implant or system may be omitted. It should also be appreciated that the pedicle screw assemblies described herein need not be limited to use in the pedicle. For example, the self-expanding element can be used with other types of bone screws implanted elsewhere in the body where pull-out failure might be a problem.

In one aspect, the self-expanding element is associated with a bone screw that is initially delivered into the bone in a constrained configuration and subsequently deployed, such that the overall resistance of the associated bone screw to displacement is enhanced. The deployment can be achieved by removing a constraining element. The constraining element can be tubular or generally tubular and the self-expanding element can radially expand from the constrained configuration to the deployed configuration. The bone screw can be associated with the self-expanding element after deployment. The bone screw can be thread associated with the self-expanding element and/or at least one attachment feature. The attachment feature can be located distal to the self-expanding element. In an interrelated aspect, a method is described in which the deployed self-expanding element is manipulated and/or positioned after deployment to further optimize bone screw resistance to displacement. The manipulation can include pulling the device along the axis of delivery. The bone screw can be associated with the self-expanding element after manipulation.

In an interrelated aspect, disclosed is a bone screw associated with a self-expanding element that is initially delivered into the bone with the self-expanding element radially constrained and subsequently deployed into a radially expanded configuration relative to the long axis of the bone screw. The bone screw can be thread associated with the self-expanding element and/or at least one attachment feature after deployment into the radially expanded configuration. In an interrelated aspect, the bone screw of any of the devices, assemblies or methods provided herein can be a pedicle screw for fixation to the spine.

In an interrelated aspect, disclosed is a pedicle screw device having a modular association of the receiver element to the pedicle screw threaded shaft via a respectively associated tapered shaft and tapered bore providing an interference fit that is secured with a compression screw or interference pin or screw. The relative position of rotation along the long axis of the screw shaft can be infinitely adjustable. The modular receiver elements associated with the pedicle screw threaded shaft can be different lengths.

In an interrelated aspect, disclosed is a modular pedicle screw device having a receiver element that is associated with modular inserts below and above the connecting rod or element providing increased surface areas in contact between the receiver element inserts and the connecting element or rod with final receiver element engagement. The channel axis can be generally defined by the modular inserts and can be between 89 degrees and 121 degrees relative to the long axis of the pedicle screw threaded shaft. A set screw can be associated with the upper modular insert.

In an interrelated aspect, disclosed is a pedicle screw device having one or more features selected from a modular association of the receiver element to the pedicle screw threaded shaft, a relative position of rotation along the long axis of the screw shaft that can be infinitely adjustable, receiver elements associated with the pedicle screw threaded shaft can have different lengths, one or more modular inserts can be included above and/or below the connecting rod, and a self-expanding element. In an interrelated aspect, disclosed is a self-expanding pedicle screw assembly having one or both of a modular receiver element and one or more inserts.

In an interrelated aspect, disclosed is a bone implant having a spontaneously expanding or self-expanding element that is advanced along a linear axis into a bone, in an initially radially constrained configuration and subsequently relieved of constraint to allow for radial expansion such that the overall resistance of the implant to displacement along that same linear axis is increased. The bone screw, bolt, or other fixation means, can be attached to the self-expanding element in its radially expanded condition. The constraining element can be tubular or generally tubular and the self-expanding element can be generally spindle or cylindrically configured in the radially expanded condition. The shape or profile of the expanded self-expanding element can be spherical, oblong, square, rectangular, cylindrical, or any of a variety of other shapes. The bone screw, bolt, or other bone fixation means can be thread associated with the self-expanding element and/or an associated attachment feature. The bone implant can be deployed into a boney structure and the self-expanding element following radial expansion, can be displaced from an initial position to subsequent position along the same insertion axis. The displacement of the self-expanding element can include pulling the bone implant along the axis of insertion with a dissociable and thread attached element. A bone screw, bolt, or other bone fixation means, can be associated with the self-expanding element after displacement.

In an interrelated aspect, disclosed is a bone implant having a bone screw associated with a self-expanding element that can be initially delivered into the bone with the self-expanding element radially constrained and subsequently deployed into a radially expanded configuration relative to the axis of insertion. The bone screw can be thread associated with the self-expanding element and/or at least one attachment feature after deployment into the radially expanded configuration. The bone screw can be a pedicle screw for fixation to the spine. The pedicle screw can have a modular association of the receiver element to the pedicle screw shaft via close conforming tapered conical features associated with the modular receiver element and the pedicle screw shaft. The pedicle screw can include a mating conical tapered feature of the modular receiver element that is axially compressed into the conical tapered bore of the pedicle screw shaft using a compression screw. The relative position of rotation of the modular receiver element to the pedicle screw shaft can be infinitely adjustable. Different axial lengths of modular receiver elements can be associable to the pedicle screw threaded shaft. The connecting rod channel axis can define an angle relative to the pedicle screw axis that is between 89 degrees and 121 degrees. The compression screw can be delivered through a cannulated feature in the modular receiver element and threaded to an internal threaded bore in the pedicle screw shaft

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

1. A fixation system for a boney structure, comprising:
a bone screw; and
a self-expanding element configured to be associated with the bone screw, wherein the self-expanding element has a narrow constrained configuration during delivery and an expanded deployed configuration after delivery that increases resistance of the bone screw to displacement.

2. The fixation system of claim 1, further comprising a containment sleeve, wherein deployment is achieved by removing the containment sleeve.

3. The fixation system of claim 2, wherein the containment sleeve is generally tubular and the self-expanding element radially expands from the constrained configuration to the deployed configuration.

4. The fixation system of claim 1, wherein the bone screw is associated with the self-expanding element after deployment.

5. The fixation system of claim 1, wherein the bone screw is thread associated with the self-expanding element and/or at least one attachment feature.

6. The fixation system of claim 5, wherein the at least one attachment feature is located distal to the self-expanding element.

7. The fixation system of claim 1, wherein the bone screw is a pedicle screw for fixation to the spine.

8. A method of treating a boney structure with a fixation device, wherein the fixation device comprises a bone screw and a self-expanding element configured to be associated with the bone screw, the method comprising:
deploying the bone screw in the boney structure;
deploying the self-expanding element to be associated with the bone screw; and
optimizing resistance to displacement of the bone screw by manipulating the deployed self-expanding element.

9. The method of claim 8, wherein manipulation comprises pulling the fixation device along an axis of delivery of the bone screw.

10. The method of claim 9, wherein the bone screw is associated with the self-expanding element after manipulation.

11. The method of claim 8, wherein the bone screw is a pedicle screw for fixation to the spine.

12. A fixation system for a boney structure, comprising:
a bone screw associated with a self-expanding element, wherein the bone screw is deliverable into the boney structure with the self-expanding element radially constrained and wherein the self-expanding element is configured to be subsequently deployed into a radially expanded configuration relative to the long axis of the bone screw.

13. The fixation system of claim 12, wherein the bone screw is thread-associated with the self-expanding element and/or at least one attachment feature after deployment into the radially expanded configuration.

14. The fixation system of claim 12, wherein the bone screw is a pedicle screw for fixation to the spine.

15. A fixation device comprising:
a pedicle screw having a threaded shaft; and
a receiver element modularly associated with the threaded shaft via a tapered shaft and tapered bore providing an interference fit; and
a locking element configured to secure the interference fit.

16. The device of claim 15, wherein the locking element is a compression screw, an interference pin or a screw.

17. The device of claim 15, wherein a relative position of rotation along the long axis of the threaded shaft is infinitely adjustable.

18. The device of claim 15, wherein the modular receiver element is selected from a plurality of receiver elements having different lengths.

19. A modular bone screw system, comprising:
a bone screw having a threaded shaft;
a connecting element;
a plurality of modular spacers; and
a receiver element,
wherein the receiver element is configured to be associated with one of the plurality of modular spacers below and one of the plurality of modular spacers above the connecting element providing increased surface area contact between the spacers and the connecting element with final receiver element engagement.

20. The system of claim 19, wherein an axis of a channel is generally defined by the plurality of modular spacers and is between 89 degrees and 121 degrees relative to the long axis of the threaded shaft.

21. The system of claim 19, further comprising a set screw associated with modular spacer positioned above the connecting element.

22-40. (canceled)
